(19) United States
(12) Patent Application Publication
(10) Pub. No.: US 2024/0133508 A1
(43) Pub. Date: Apr. 25, 2024

(54) MINERAL WOOL INSULATION

(71) Applicants: KNAUF INSULATION SPRL, Vise (BE); KNAUF INSULATION, INC., Shelbyville, IN (US)

(72) Inventors: Carl HAMPSON, Liverpool (GB); Gert MUELLER, New Albany, OH (US); Charles APPLEY, Cumberland, IN (US)

(21) Appl. No.: 18/399,588

(22) Filed: Dec. 28, 2023

Related U.S. Application Data
(63) Continuation of application No. 18/382,518, filed on Oct. 22, 2023.
Application No. 18/382,518 is a continuation of application No. 18/231,931, filed on Aug. 9, 2023.
Application No. 18/231,931 is a continuation of application No. 18/199,966, filed on May 21, 2023, now abandoned.
Application No. 18/199,966 is a continuation of application No. 18/117,591, filed on Mar. 6, 2023, now abandoned.
Application No. 18/117,591 is a continuation of application No. 18/074,643, filed on Dec. 5, 2022, now abandoned.
Application No. 18/074,643 is a continuation of application No. 17/896,320, filed on Aug. 26, 2022, now abandoned.
Application No. 17/896,320 is a continuation of application No. 15/943,055, filed on Apr. 2, 2018, now abandoned.
Application No. 15/943,055 is a continuation of application No. 15/265,120, filed on Sep. 14, 2016, now abandoned.
Application No. 15/265,120 is a continuation of application No. 12/671,922, filed on Apr. 15, 2011, now Pat. No. 9,469,747.

(30) Foreign Application Priority Data
Aug. 2, 2007 (GB) 0715100.4
Apr. 28, 2008 (GB) 0807777.8
Jun. 5, 2008 (GB) 0810297.2

(51) Int. Cl. F16L 59/02; C03C 13/06; C03C 25/32; C03C 25/321; C03C 25/34; C08H 8/00; C08K 5/17; C08K 7/14; C08L 97/02; C08L 99/00; C08L 101/02; C08L 101/06; C09D 197/02; C09D 199/00; C09D 201/02; C09D 201/06; C09K 3/00; D04H 1/4209; D04H 1/4218; D04H 1/64; E04B 1/76

(52) U.S. Cl. CPC F16L 59/028; C03C 13/06; C03C 25/32; C03C 25/321; C03C 25/34; C08H 8/00; C08K 5/175; C08K 7/14; C08L 97/02; C08L 99/00; C08L 101/025; C08L 101/06; C09D 197/02; C09D 199/00; C09D 201/025; C09D 201/06; C09K 3/00; D04H 1/4209; D04H 1/4218; D04H 1/64; E04B 1/7658; E04B 1/7662

(57) ABSTRACT

A method of manufacturing a mineral fibre thermal insulation product comprises the sequential steps of:
Forming mineral fibres from a molten mineral mixture;
Spraying a substantially formaldehyde free binder solution on to the mineral fibres, the binder solution comprising: a reducing sugar, an acid precursor derivable from an inorganic salt and a source of nitrogen;
Collecting the mineral fibres to which the binder solution has been applied to form a batt of mineral fibres; and
Curing the batt comprising the mineral fibres and the binder which is in contact with the mineral fibres by passing the batt through a curing oven so as to provide a batt of mineral fibres held together by a substantially water insoluble cured binder.

### CROSS-REFERENCE TO RELATED APPLICATIONS

This application is a continuation of U.S. application Ser. No. 12/671,922, filed Apr. 15, 2011, which is a U.S. national counterpart application of International Application Serial No. PCT/EP2008/060178, filed Aug. 1, 2008 under 35 U.S.C. § 371, which claims priority to GB Patent Application Serial Number 0715100.4, filed Aug. 3, 2007, GB Patent Application Serial Number 0807777.8, filed Apr. 29, 2008, and GB Patent Application Serial Number 0810297.2, filed Jun. 6, 2008, the entire disclosure of each of which is hereby incorporated herein by reference.

### TECHNICAL FIELD

This invention relates to the manufacture of mineral wool insulation, for example glass wool or stone wool insulation, and to mineral wool insulation products.

### BACKGROUND

WO 2007/014236 (incorporated herein by reference) discloses manufacture of mineral wool insulation products using binders which comprise Maillard reactants. One particular binder disclosed is based on a triammonium citrate-dextrose system derived from mixing dextrose monohydrate, anhydrous citric acid, water and aqueous ammonia. One of the many advantages of this binder system is that it is formaldehyde free.

### SUMMARY

One aspect of the present invention provides a method of manufacturing a mineral fibre thermal insulation product in accordance with claim 1; further aspects of the inventions are defined in other independent claims. The dependent claims define alternative and/or preferred embodiments.

Binder solutions used in accordance with the present invention may be “substantially formaldehyde free”, that is to say that they liberate less than 5 ppm formaldehyde as a result of drying and/or curing (or appropriate tests simulating drying and/or curing). Such binder solutions are preferably “formaldehyde free”, that is the say they liberate less than 1 ppm formaldehyde in such conditions.

Insulation materials in accordance with the invention which incorporate binders may be “substantially formaldehyde free”, that is to say that they comprise less than 5 ppm or less than detectable limits of free formaldehyde and/or consist of materials which together comprise less than these amounts of free formaldehyde and/or release levels of formaldehyde in standardised tests adapted to simulate their ordinary use which allows them to be classified as having no or undetectable levels of formaldehyde release. Preferably, such products release less than 10 μg/m³, more preferably less than 5 μg/m³ of formaldehyde during the period of 24-48 hours from the start of testing in accordance with ISO 16000.

### BRIEF DESCRIPTION OF THE DRAWINGS

FIG. 1 shows a plan view of a mineral fibre test sample, where r is radius=12.7 mm, DC is distance between centers=44.5 mm, a=25.4 mm, and b=121 mm.

### DETAILED DESCRIPTION

It has been found that insulation materials made according to the present invention may have at least equivalent and indeed improved properties compared to, for example, products made using the tri-ammonium citrate-dextrose system of WO 2007/014236. WO 2007/014236 teaches binder systems based, inter alia, on a combination of a carbohydrate (for example a reducing sugar), ammonia and a carboxylic acid and suggests that a Maillard type reaction may form the basis of the curing chemistry. It would have been thought that the nature of the acid used would have a significant effect upon the properties of the cured binder, particularly if the acid precursor and/or a derivative therefrom is incorporated into the structure of the cured binder. It is thus surprising that an acid precursor derivable from an inorganic salt should provide a suitable acid precursor in an otherwise apparently similar binder system.

Use of an acid precursor derivable from an inorganic salt may have significant advantages in terms of cost, availability and ease of handling. A particular advantage can be achieved by use of one or more inorganic ammonium salts, for example, an ammonium sulphate, an ammonium phosphate or an ammonium carbonate. An ammonium salt may provide the or part of the acid precursor and/or the or part of the source of nitrogen and/or the or part of a pH control system. An ammonium nitrate may also work; however, ammonium nitrate may oxidise aldehyde groups of the carbohydrate (for example in the case of dextrose) and/or require precautions to avoid explosions.

An ammonium sulphate is particularly advantageous but ammonium phosphate may be used in addition to or instead of this. Ammonium phosphate may be mono ammonium phosphate, di ammonium phosphate or tri ammonium phosphate; it may be an ammonium hydrogen phosphate. An ammonium carbonate, alone or in combination with the other materials disclosed herein, may also provide good results. The ammonium carbonate may be an ammonium bicarbonate.

The acid precursor, particularly when this consists essentially of inorganic ammonium salt(s), may make up
at least 5%, preferably at least 7%, more preferably at least 9% by dry weight of the uncured binder solution; and/or
less than 20%, preferably less than 18%, more preferably less than 16% by dry weight of the uncured binder solution.

The acid may comprise: a sulphuric acid, a phosphoric acid, a nitric acid or a weak acid.

The binder may comprise between 5%-25%, preferably 10% to 20%, more preferably 15% to 20% by dry weight of acid precursor (particularly where this is an inorganic ammonium salt) to carbohydrate (particularly when this is a sugar).

Where the binder comprises both an acid precursor derivable from an inorganic salt and an organic acid with the carbohydrate (particularly where this is a sugar), these may be present in the following amounts by dry weight with respect to the carbohydrate:

| | Preferred | More preferred | Most preferred |
|---|---|---|---|
| acid precursor | At least 2.5% | At least 5% | |
| derivable from an | | | |
| inorganic salt | | | |
| organic acid | At least 2.5% | At least 5% | |
| Combination of | 5-25% | 10-20% | 15-20% |
| organic acid and | | | |
| acid precursor | | | |
| derivable from an | | | |
| inorganic salt | | | |

Where an organic acid is used, this is preferably derived from an ammonium salt. For example, an ammonium citrate, particularly tri-ammonium citrate may be used as a source of citric acid.

Prior art phenol formaldehyde binder systems for mineral wool insulation have been used with the addition of about 2% by weight ammonium sulphate as a curing agent. However, the chemistry of such phenol formaldehyde binder systems is not comparable to the binder systems of the present invention which are not based on phenol and/or formaldehyde and/or on other phenolics.

A carbohydrate may be used in the binder solution rather than specifically a reducing sugar and may comprise a monosaccharide, for example in its aldose or ketose form. Preferably, the carbohydrate comprises a sugar, more preferably a reducing sugar or a reactant that yields a reducing sugar in situ under thermal curing condition; it may comprise glucose (ie dextrose). The carbohydrate may comprise a carbohydrate having a reducing aldehyde. It is believed that the use of a reducing sugar and particularly dextrose gives particularly good results for the manufacture of mineral wool insulation products. The dextrose need not be 100% pure but use of a material having a dextrose equivalent value of at least 0.85, preferably at least 0.9 and more preferably at least 0.95 is thought to be advantageous. The dextrose equivalent value DE can be thought of as i) a measure of de-polymerization and is roughly: DE=100/dp where dp stands for degree of polymerization or ii) the total amount of reducing sugars calculated as D-glucose (dextrose) on a dry basis.

Preferably, the binder solution and/or the binder is free or substantially free of starch; the presence of substantial quantities of starch is thought to increase the curing time and/or reduce the strength of the cured binder. The binder solution and/or the binder may be free or substantially free of proteins.

Industrial, non-food grade dextrose may be used as the reducing sugar; products such as Sirodex331 which is a 75% solids sugar solution obtainable from Tate and Lyle with a DE value of 94.5 may be used.

Particularly in the case where the reducing sugar consists essentially of dextrose and the acid precursor consists essentially of an ammonium salt, for example an ammonium sulphate, the ratio by dry weight of the amount of reducing sugar/the amount of acid precursor may be greater than or equal to 2.5 and/or less than or equal to 13.

The source of nitrogen may be an amine or an amine reactant; it may be derivable from the same source as the acid precursor, for example, from an inorganic ammonium salt. It is preferably ammonia in solution.

Precursors for and/or reactants which give the materials referred to may be used.

In one embodiment, the binder is derived essentially from a reducing sugar and an inorganic ammonium salt in aqueous solution.

In another embodiment, the binder may also comprise an organic acid, particularly a carboxylic acid; this may be a polycarboxylic acid, particularly a bi-carboxylic acid or tri-carboxylic acid, preferably citric acid; it is preferably monomeric. The combination of an organic acid (or a precursor a salt or an anhydride thereof) with an acid precursor derivable from an inorganic salt may present various advantages. Firstly, such a combination may reduce the risk of punking (which has been observed with such binders based solely on organic acids) whilst providing acceptable strength. Punking is a term of art in the mineral fibre insulation area which generally denotes a comparatively rapid oxidation of a binder with a concomitant generation of heat in a finished and generally packaged insulation product. Punking generally causes generation of fumes and discolouring of the insulation material. It may be associated with exothermic reactions which increase the temperatures through the thickness of the insulation material; this may destroy the integrity of the insulation product and/or present a fire hazard.

Alternatively or additionally, the combination of an organic acid (or a precursor, a salt or an anhydride thereof) with an acid precursor derivable from an inorganic salt may moderate acid conditions occurring during curing and thus reduce the risk or tendency of such conditions to cause significant damage to the material being bound. Such a combination may be particularly advantageous as a binder for stone wool insulation whose fibres may be more susceptible to potential damage by acid than, for example, glass wool insulation.

In a further embodiment, the binder is derived essentially from: a carbohydrate; an inorganic ammonium salt; and an organic acid and/or organic acid precursor; in aqueous solution.

The term “consist or consisting essentially of” is intended to limit the scope of a claim to the specified materials or steps and those that do not materially affect the basic and novel characteristic(s) of the claimed invention.

Binders which comprise or consist essentially of the components described herein may include additives, for example, additives selected from: silanes, mineral oils, coupling agents, silicones or siloxanes (particularly for water repellency), silicon containing compounds, surfactants, hydrophilic additives, hydrophobic additives, waxes, substances useful for controlling the pH (e.g. ammonium hydroxide) and ammonia. Ammonium hydroxide when used, and indeed other additives, may provide the and/or an additional source of nitrogen. Preferably, the total quantity of additives (excluding ammonia) is less than 5% by weight (excluding the weight of water present), more preferably less than 3% or less than 2% by weight.

It is preferred to include a silane as an additive. The binder and/or binder solution may comprise at least 0.1% and/or less than 1% of a silane by dry weight. The silane may be amino substituted; it may be a silyl ether and it is believed that its presence may significantly improve the long term strength of the binder, particularly after weathering.

Preferences for the pH of the binder are:

| | Preferred | More preferred | Most preferred |
|---|---|---|---|
| pH of binder | ≥7 | ≥8 | ≥9 |

at least in the state in which the binder is applied to a material to be bound and/or recovered in a waste water recuperation system. Such a neutral or alkaline pH of the binder may alleviate problems of corrosion of manufacturing equipment which have been encountered with some essentially acidic prior art binder systems. Such prior art binders include binders consisting essentially of polyacrylic acids or polymer polycarboxylic acids. One particular advantage of the present invention is thus the use of a binder system that can operate in such neutral or alkaline conditions. When cured, the binder may become acidic during the curing process. However, equipment corrosion considerations are less significant in this case due to the minimal contact between the manufacturing equipment and the binder when in this state. The pH of the binder may be less than or equal to 13, preferably less than or equal to 12, 11 or 10. A preferred pH may be in the range of 7.5 to 9.5, particularly 8 to 9.

It is preferred to arrange the pH of the binder solution at an appropriate level to prevent precipitation of its constituents and particularly to ensure that the acid precursor derivable from an inorganic salt remains in solution. This is particularly the case where ammonium phosphate provides the acid precursor. Better dry and/or weathered strengths and/or more homogeneous products may be achieved by using homogeneous binder solutions comprising ammonium salt acid precursors which are free from precipitates, particularly when ammonium phosphate is used and the binder solution is free from phosphate precipitates.

The binder composition may be provided in the form of an aqueous solution; it may contain free ammonia or excess ammonia in solution. A neutral or alkaline pH of the binder may be generated by an excess of alkaline groups compared with acid groups present in the binder solution, for example, due partially or substantially to the presence of ammonia in the solution. Additional ammonia may be added to the binder solution, for example 0.2%-1% by weight, or indeed more; this may help to keep a wash water system used in the manufacture of mineral wool insulation alkaline over the long term.

When binder solution is sprayed on to hot mineral wool fibres just after they have been formed, the residual heat of the mineral wool fibres may cause a significant portion of any water in the binder solution to evaporate. Consequently, the mineral wool fibres which are then collected to form a batt may have binder present on them in the form of a sticky, viscous or tacky liquid. This may facilitate bonding between individual fibres via the binder.

One of the many advantages of this binder system is that it is sprayed onto the mineral wool fibers in a substantially unreacted state. The ability to spray the binder solution onto the mineral wool fibers in a substantially unreacted state may alleviate problems associated with pre-reacting the binder components in solution which have been encountered with some prior art binder systems in which the components are pre-reacted. Such prior art binders include binders consisting essentially of pre-reacted polymers or resins which are applied to the materials to be bound. With substantially unreacted binder present on the mineral wool fibers in the form of a sticky, viscous or tacky liquid, the reaction between the binder components may occur in a substantially dry state. One may describe the reaction as a bulk polymerization because it is occurring without the benefit of a solvent. A particular advantage of the present invention is thus the use of a binder system that can polymerise in a substantially dry state or through a bulk polymerisation.

The mineral fibres may be formed by internal or external spinning. They may have a temperature in the range 20° C. to 200° C., generally 30° C. to 100° C. or 150° C., when sprayed with the binder solution. The quantity of binder solution sprayed may be used with or without additional water sprays to assist in cooling the mineral fibres to a desired temperature between their formation and their collection to form a batt.

A particular advantage of using ammonia in solution to control the pH of the binder solution applied to the mineral fibres is that at least part of the ammonia of binder solution that sticks to the fibres may flash off due to the residual heat of the mineral wool fibres. Consequently, the binder solution that coats the fibres may have a lower pH than the binder solution sprayed.

The binder may be curable; it may be cured, for example in a curing oven; it may form a thermoset binder. In its cured form, the binder may: comprise melanoidins; and/or be thermoset; and/or be water insoluble or substantially water insoluble. The binder solution may be substantially colourless or white to off-white; upon curing, the binder may take on a dark colour, particularly a dark brown colour. The cured product may be dark in colour, particularly dark brown in colour. The binder may be free of proteins; it may be free of cellulosic feedstock. One of the many advantages of this binder system is that the extent of curing can be determined by the colour. Substantially dehydrated binder appears white or off-white. Progressively cured to a greater extent, the binder appears progressively darker in colour (a darker shade of brown). When applied to mineral fibers, the extent to which the mineral wool insulation has cured can be determined by its colour.

When applied to the mineral fibres and/or prior to passing through the curing oven, the binder may be free or substantially free of melanoidins and/or other reaction products derived from curing. Curing of the binder may produce glucosylamine, particularly as an intermediate product. Consequently, a cured or particularly a partially cured product may comprise glucosylamine.

The reaction of the binder upon curing may be essentially a Maillard type reaction as described for example in US Patent Application 20070027283 or WO2007/14236. The binder may comprise polymerisation products of a mixture that comprises a reducing sugar and a material selected from the group consisting of ammonium sulphate, ammonium phosphate, ammonium nitrate and ammonium carbonate.

The binder solution may be formulated by combining:
A carbohydrate, preferably a reducing sugar;
An acid precursor derivable from an inorganic salt, preferably an ammonium sulphate or ammonium phosphate;
A source of nitrogen; and
water.

The formulation may comprise optional or additional ammonia provided in the form of an aqueous ammonia solution. The water may comprise wash water.

Forming the binder solution from a carbohydrate and an acid precursor comprising an inorganic ammonium salt provides one particular advantageous preparation method. This may be achieved in a simple mixing chamber which may be open and/or at atmospheric pressure. The carbohydrate and/or the acid precursor may be added in powder or liquid form. The preparation is preferably carried out at room temperature. Preferably it is not necessary to supply heat to prepare the binder solution; nevertheless, the binder solution may be heated during its preparation, for example to a temperature with the range 20° C. to 80° C., particularly where this facilitates dissolving and/or mixing of its ingredients.

The binder solution may comprise:
at least 5% 10%, 15% or 18% solids and/or
less than 50%, 40% or 20% solids

particularly determined as bake out solids by weight after drying at 140° C. for 2 hours.

The binder solution and/or the binder are preferably organic.

The mineral fibre insulation may be shaped and/or dimensioned and/or moulded with the aid of the binder.

The binder solution, particularly when applied to the mineral fibres, may have a viscosity appropriate for application by spraying or pouring. Its viscosity at 20° C. may be:
Less than about 1.5 Pa·s, preferably less than about 1×10⁻² Pa·s; and/or
Greater that about 2×10⁻⁴ Pa·s, preferably greater than about 5×10⁻⁴ Pa·s

Curing of the binder may occur in a curing oven, for example using forced hot air circulation; it may occur in a press. Curing may comprise a dehydration of the binder; it may comprise a polymerisation. Curing may comprise a bulk polymerisation reaction. Curing may be carried out for duration of 20 minutes or less, preferably 10 minutes or less. Curing of the binder preferably occurs when the binder solution (from which water may have been evaporated) is in contact with the mineral fibres; it may occur at substantially atmospheric pressure. The curing may be a substantially dry curing, that is to say by application of dry heat and/or substantially dry or heated atmospheric air rather than using steam or heated water vapour.

The curing temperature and time may be selected as a function of the product density and/or thickness. The curing oven in such cases may have a plurality of heating zones having temperatures within the range 200° C. to 350° C. (typically 230° C. to 300° C.). A thin, low density product (12 kg/m³ or less) may be cured by passing through the curing oven in as little as 20 seconds; a thick, high density product (80 kg/m³ or more) may require a passage of 15 minutes or more in the curing oven. The product may reach a temperature in the range 180° C.-220° C. during the curing process.

The cured binder may comprise greater than 2% and/or less than 8% nitrogen by mass as determined by elemental analysis.

The binder in its uncured state may comprise the following levels of sulphates, phosphates carbonates and/or nitrates by dry weight:
Greater than 2.5%, 3% or 5%; and/or
Less than 25%, 22%, or 20%

Finished materials manufactured using binder systems according to the present invention may have residual levels of sulphates, phosphates, carbonates and/or nitrates derived notably from the inorganic salt serving as the acid precursor. Such species may be present in the following quantities:
Greater than 500, 750, 1000 or 1500 mg/kg; and/or
Less than 5000, 4000 or 3000 mg/kg.

The presence of such species may be assessed in a leach test and provide an indication in the final product of the binder system used.

The quantity of binder in the finished product may be:
Greater than: 1%, 2%, 2.5%, 3%, 3.5% or 4%; and/or
Less than: 20%, 15%, 10% or 8%

measured by dry weight of the finished product.

The mineral wool insulation may have one or more of the following parting strengths:

Ordinary Parting Strength of
At least 120 g/g, preferably at least 150 g/g; and/or
Less than 400 g/g

Weathered Parting Strength of
At least 120 g/g, preferably at least 150 g/g; and/or
Less than 400 g/g

% loss between Ordinary and Weathered Parting Strength of
Less than 10%, preferably less than 5%

The mineral wool insulation may have one or more of the following characteristics:
A density greater than 5, 8 or 10 kg/m;
A density less than 200, 180 or 150 km/m³
Comprise glass wool fibres and have a density greater than 5, 8 or 10 kg/m³ and/or less than 80, 60 or 50 kg/m³;
Comprise stone wool fibres and have a density greater than 15, 20 or 25 kg/m³ and/or less than 220, 200 or 180 kg/m³;
A thermal conductivity λ of less than 0.05 W/mK and/or greater than 0.02 W/mK
Comprise less than 99% by weight and/or more than 80% by weight mineral fibres.
A thickness of greater than 10 mm, 15 mm or 20 mm and/or less than 400 mm, 350 mm or 300 mm.

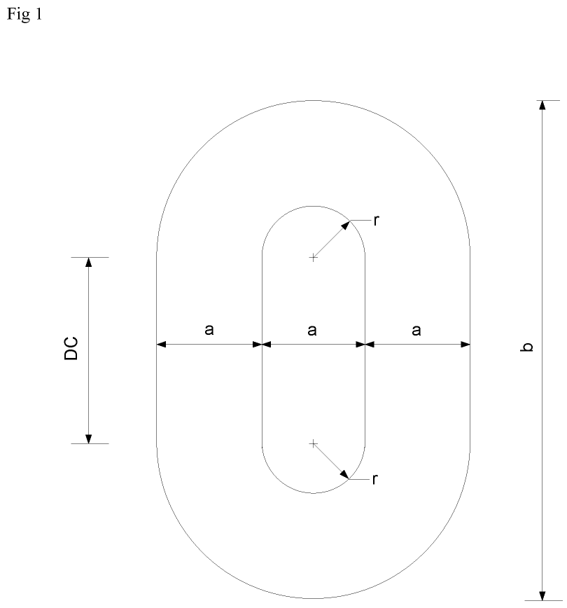

Embodiments of the invention will now be described by way of example with reference to FIG. 1 which is a plan view of a test sample.

### Shell Bone Testing:

Binders were prepared as aqueous solutions by
combining the ingredients of a desired binder formulation in an open, unheated reaction vessel
adding distilled water
subsequently adding a silane solution
agitating during addition of liquids and afterwards for several minutes to achieve complete dissolution of solids

such that the binder solution contained approximately 45% dissolved solids as a percentage of total weight of solution. A 2-g sample of this solution, upon thermal curing at about 200° C. to 210° C. for 8 minutes, would yield 30% solids (the weight loss being attributed to dehydration during thermoset binder formation).

An evaluation of dry and “weathered” tensile strength of glass bead-containing shell bones provided an indication of the likely tensile strength and the likely durability of fibreglass insulation or other materials prepared with that particular binder. Predicted durability is based on the ratio of a shell bone's weathered tensile strength to its dry tensile strength.

To prepare the shell bones, an electric mixer was used for about two minutes to mix approximately 75 g of binder with 727.5 g of glass beads (equivalent to Quality Ballotini Impact Beads, Spec. AD, US Sieve 70-140, 106-212 micron-#7, from Potters Industries, Inc.). Any clumps from the sides of the mixer whisk and from the sides and bottom of the mixing bowl were mixed in manually using a spatula about half way through the mixing and also at the end of the mixing.

The prepared glass beads/binder mixture was added to the mould cavities of a shell bone mould (Dietert Foundry Testing Equipment; Heated Shell Curing Accessory, Model 366) which had been pre-heated to about 218° C. (425° F.). The surface of the mixture in each cavity was flattened out, while scraping off the excess mixture to give a uniform surface area to the shell bone. Any inconsistencies or gaps that existed in any of the cavities were filled in with additional glass beads/binder mixture and then flattened out. The top platen was quickly placed onto the bottom platen (to avoid producing shell bones with two differentially cured layers). The cured shell bones were removed after seven minutes, cooled to room temperature on a wire rack, labelled and placed individually in plastic storage bags. If shell bones could not be tested on the day they were prepared, the shell bone-containing plastic bags were placed in a dessiccator unit. During curing the temperature of the bottom platen ranged from about 204° C. to about 221° C. (about 400° F. to about 430° F.), while the temperature of the top platen ranged from about 227° C. to about 243° C. (about 440° F. to about 470° F.).

### Procedure for Testing Breaking Strength:

Equipment: 5500 R Instron machine
Immediately prior to testing, each shell bone was removed from is plastic bag and its weight and thickness recorded.

### Weathering Procedure for Shell Bones:

16 hours weathering in a pre-heated humidity chamber (65° C., 95% relative humidity)
upon removal shell bones were sealed in individual plastic storage bags and taken immediately for testing.

### Procedure for Measuring Gel Time:

A small amount of binder (2.0 ml) is added to the centre of a hot plate set to 150° C. and a stop watch is started. The binder is worked with a spatula until it is possible to draw the sample into a long string. The time taken from the addition of the binder to the string formation is the gel time.

Binder Formulations Tested—Inorganic Acid Precursors Compared with Citric Acid:

| Test ref: | Binder formulation (by dry weight) |
|---|---|
| A | 85% DMH + 15% CA + 4.8% NH4OH + 0.3% ISI0200 |
| B | 90% DMH + 10% AmSO4 + 4.8% NH4OH + 0.3% ISI0200 |
| C | 85% DMH + 15% AmSO4 + 4.8% NH4OH + 0.3% ISI0200 |
| D | 80% DMH + 20% AmSO4 + 4.8% NH4OH + 0.3% ISI0200 |
| E | 90% DMH + 10% AmPO4 + 4.8% NH4OH + 0.3% ISI0200 |
| F | 85% DMH + 15% AmPO4 + 4.8% NH4OH + 0.3% ISI0200 |
| G | 80% DMH + 20% AmPO4 + 4.8% NH4OH + 0.3% ISI0200 |

Binder Formulations Tested—Combined Inorganic Acid Precursor and Citric Acid Compared with Citric Acid Alone and Inorganic Acid Precursor Alone:

| Test | |
|---|---|
| ref: | Binder formulation (by dry weight) |
| H | 85% DMH + 15% CA + 4.8% NH4OH + 0.3% ISI0200 |
| I | 85% DMH + 10% CA + 5% AmSO4 + 4.8% NH4OH + 0.3% |
| | ISI0200 |
| J | 85% DMH + 5% CA + 10% AmSO4 + 4.8% NH4OH + 0.3% |
| | ISI0200 |
| K | 85% DMH + 15% AmSO4 + 4.8% NH4OH + 0.3% ISI0200 |

Key:
DMH = Dextrose monohydrate
CA = citric acid
NH4OH = ammonium hydroxide
ISIO200 = silane
AmSO4 = ammonium sulphate
AmPO4 = ammonium phosphate

Test Results—Inorganic Acid Precursors Compared with Citric Acid:

| | | | Loss in | | pH of binder |
|---|---|---|---|---|---|
| | Dry | Weathered | breaking | | solution |
| | breaking | breaking | strength | Gel time | just before |
| Test | strength | strength | from weath- | of binder | mixing |
| ref | (MN/m²) | (MN/m²) | ering/% | solution (s) | with beads |
| A | 1.455 | 1.567 | −7.70 | 343 | 9.54 |
| B | 1.271 | 0.895 | 29.57 | 280 | 10.28 |
| C | 1.550 | 0.856 | 44.79 | 362 | 10.24 |
| D | 1.877 | 1.156 | 38.39 | 327 | 10.13 |
| E | 1.499 | 1.069 | 28.68 | 356 | 10.18 |
| F | 1.281 | 0.848 | 33.82 | 334 | 9.99 |
| G | 1.123 | 0.801 | 28.74 | 287 | 9.73 |

Test Results—Combined Inorganic Acid Precursor and Citric Acid Compared with Citric Acid Alone and Inorganic Acid Precursor Alone:

| | | | Loss in | | pH of binder |
|---|---|---|---|---|---|
| | Dry | Weathered | breaking | | solution |
| | breaking | breaking | strength | Gel time | just before |
| Test | strength | strength | from weath- | of binder | mixing |
| ref | (MN/m²) | (MN/m²) | ering/% | solution (s) | with beads |
| H | 1.69 | 1.50 | 11.32 | 363 | 9.39 |
| I | 1.50 | 1.18 | 21.37 | 341 | 9.71 |
| J | 1.21 | 1.05 | 13.19 | 375 | 9.99 |
| K | 1.47 | 1.02 | 30.33 | 376 | 9.97 |

Results from tests carried out together (test A to G were carried out in one session and tests H to K carried out during another session) provide a useful indication of results relative to other results obtained during the same test session. It may not be reliable to compare tests results from different test sessions.

### First Comparative Testing on Insulation Product:

Comparative testing of binder systems on a mineral fibre insulation product gave the following results:

| Binder | | |
|---|---|---|
| tested | Description | Formulation |
| PF1 | Comparative example - standard | Resin, Urea, Lignin, |
| | phenol formaldehyde binder | Ammonia, Silane |
| AC1 | Comparative example - | Dextrose 85% Citric Acid |
| | ammonium citrate based binder | 15% Ammonia 4.8% Silane |
| | | 0.3% |
| Ex1 | Example 1 of the present | Dextrose 85% Ammonium |
| | invention | Sulphate 15% Ammonia 4.8% |
| | | Silane 0.3% |

| | |
|---|---|
| Product used | glass wool fibre insulation product, nominal density 16 |
| for test: | kg/m³, nominal thickness 75 mm, nominal width 455 mm |

### Binder Content of Test Product LOI (Loss on Ignition) % Weight:

| Binder | Mean LOI |
|---|---|
| PF1 | 6.22% |
| AC1 | 6.91% |
| Ex1 | 6.78% |

Drape Test (Mean Average in Mm Measured after the Periods Specified):

| Binder | Day 1 | Week 1 | Week 3 | Week 6 |
|---|---|---|---|---|
| PF1 | 55 | 68 | 60 | 71 |
| AC1 | 83 | 99 | 80 | 72 |
| Ex1 | 66 | 76 | 66 | 75 |

Thickness (Mean Average in Mm Measured after the Periods Specified in Accordance with British Standard BS EN 823:1995

| Binder | Day 1 | Week 1 | Week 3 | Week 6 |
|---|---|---|---|---|
| PF1 | 76.4 | 75.1 | 75.1 | 75.2 |
| AC1 | 75.3 | 73.6 | 72.5 | 74 |
| Ex1 | 76 | 76.7 | 74.9 | 74.3 |

Density (Mean Average in Kg/m³ Measured after the Periods Specified)

| Binder | Day 1 | Week 1 | Week 3 | Week 6 |
|---|---|---|---|---|
| PF1 | 16.44 | 16.7 | 16.35 | 16.44 |
| AC1 | 16.68 | 16.41 | 16.33 | 16.48 |
| Ex1 | 16.5 | 16.9 | 16.5 | 16.5 |

### Quantity of Sulphates Present Mg/Kg

| Binder | Sample 1 | Sample 2 |
|---|---|---|
| AC1 | 240 | 240 |
| Ex1 | 2000 | 2200 |

Parting Strength (g/g)

| Binder | Ordinary | Weathered | % loss |
|---|---|---|---|
| PF1 | 248 | 107 | 56.85 |
| AC1 | 230 | 199 | 13.47 |
| Ex1 | 196 | 189 | 3.57 |

### Test Procedures:

### Binder Content LOI (Loss on Ignition)

A weighed sample of wool plus binder is placed in a muffle furnace set to 550° C. After a set time the wool is removed from the furnace, placed in a desiccator to cool and re-weighed. The weight loss is expressed as a percentage of the original sample weight and is known as the binder content or Loss On Ignition (LOI).

### Drape Test

A single batt (or slab) is placed across two poles (each 500 mm long, 20 mm diameter) set into a wall 1 metre apart. The degree of sag in the centre of the batt is recorded. This is repeated for all of the batts in a pack and for several packs. Packs are measured at set points over a period of time to determine the long term effects of compression on the batts.

Density: measured for the samples subjected to the drape test

Quantity of sulphates present: leaching test for granular wastes in water with eluate analysis according to British standard BS EN 12457-2 at L/S10

### Parting Strength

The parting strength is expressed in grams/gram being the total breaking load of six test specimens divided by their total weight.

The test is carried out on mineral fibre mats as received for testing (Ordinary Parting Strength) and after an accelerated weathering test as explained below (Weathered Parting Strength).

A first set of six samples of the form and dimensions shown in FIG. 1 are cut from the mineral fibre mat to be tested. The dimensions are:
r: radius 12.7 mm;
DC: distance between centres 44.5 mm;
a: 25.4 mm;
b: 121 mm.

The long axis of the samples should be parallel to the conveyor direction and the samples should be taken across the full width of the mineral mat. A second set of six samples is then taken in the same way.

The total weight of the first group of six samples W1 in grams is recorded.

The total weight of the second group of six samples W2 in grams is recorded; these samples are then placed in a preheated autoclave and conditioned on a wire mesh shelf away from the bottom of the chamber under wet steam at 35 kN/m² for one hour. They are then removed, dried in an oven at 100° C. for five minutes and tested immediately for parting strength.

To test the parting strength, each sample is mounted in turn on the jaws of a 5500 Instron tensile strength machine and the maximum breaking load in grams or Newtons is recorded. If the breaking load is measured in Newtons it is converted to grams by multiplying it by 101.9. Six results in grams are obtained for each set of samples: G1 G2 G3 G4 G5 and G6 for the first set of samples and G7 G8 G9 G10 G11 and G12 for the second set of samples. The Ordinary Parting Strength is calculated from the first set ofsamples usingthe formula Ordinary Parting Strength=(G1+G2+G3+G4+G5+G6)/W1.

The Weathered Parting Strength is calculated from the second set of samples using the formula Weathered Parting Strength=(G7+G8+G9+G10+G11+G12)/W2.

### Second Comparative Testing on Insulation Product:

| | |
|---|---|
| Product used | glass wool fibre insulation product, nominal density 7.2 |
| for test: | kg/m³, nominal thickness 159 mm |

SAMPLES: The following samples of fibreglass batts were tested:

| | | Target binder |
|---|---|---|
| Exam- | | content (LOI) |
| ple | Binder Description | for product |
| PF2 | standard phenol formaldehyde binder of Resin, | 4.5% |
| | Urea, Ammonia, Silane | |
| 2.1 | Dextrose 85% Ammonium Sulphate 15% | 4.5% |
| | Silane 0.3% (10.6% solids in binder solution) | |
| 2.2 | Dextrose 85% Ammonium Sulphate 15% | 4.5% |
| | Silane 0.3% Norjohn oil (11.4% solids in | |
| | binder solution) | |
| 2.3 | Dextrose 85% Ammonium Sulphate 15% | 4.5% |
| | Silane 0.3%, 2.4%NH3 (10.6% solids in | |
| | binder solution) | |
| 2.4 | Dextrose 85% Ammonium Sulphate 15% | 6.0% |
| | Silane 0.3%, 2.4%NH3 (10.6% solids in | |
| | binder solution) | |

### Results

| | PF2 | | 2.1 | | 2.2 | | 2.3 | | 2.4 | |
|---|---|---|---|---|---|---|---|---|---|---|
| Recovery | 158 | mm | 157 | mm | 163 | mm | 160 | mm | 166 | mm |
| Recovery. % | 99.4% | | 99.0% | | 102.8% | | 100.6% | | 104.8% | |
| nominal | | | | | | | | | | |
| Parting Strength | 190.8 | g/g | 131.7 | g/g | 146.7 | g/g | 159.9 | g/g | 143.9 | g/g |
| (ASTM C-686) | | | | | | | | | | |
| Parting strength | 145.9 | g/g | 100.0 | g/g | 110.3 | g/g | 124.9 | g/g | 114.3 | g/g |
| after weathering | | | | | | | | | | |
| (ASTM C-686 | | | | | | | | | | |
| following | | | | | | | | | | |
| conditioning for | | | | | | | | | | |
| 7 days at 90° F., | | | | | | | | | | |
| 90% relative | | | | | | | | | | |
| humidity) | | | | | | | | | | |

1. A method of manufacturing a mineral fibre thermal insulation product comprising the sequential steps of:
forming mineral fibres from a molten mineral mixture;
spraying a substantially formaldehyde free binder solution on to the mineral fibres, the binder solution comprising: a reducing sugar, an acid precursor derivable from an inorganic salt and a source of nitrogen;
collecting the mineral fibres to which the binder solution has been applied to form a batt of mineral fibres; and
curing the batt comprising the mineral fibres and the binder by passing the batt through a curing oven so as to provide a batt of mineral fibres held together by a substantially water insoluble cured binder.

2.-44. (canceled)

45. The method of claim 1 comprising at least one of the following features:
in which wash water is sprayed on to the mineral fibres between their formation and their collection to form a batt, at least a part of the wash water having been sprayed on mineral fibres and subsequently returned to a wash water system to be reused as wash water.
in which the binder solution is sprayed on to the mineral fibres when the mineral fibres are at a temperature of between 30° C. and 150° C.
in which the binder solution has a pH of greater than 7 when sprayed on to the mineral fibres.

46. The method of claim 1 in which curing of the binder is carried out by passing the batt through at least one zone of a curing oven at a temperature within the range 230° C.-300° C. with an oven residence time in the range 30 seconds to 20 minutes.

47. The method of claim 1 comprising at least one of the following features:
in which the acid precursor makes up between 5% and 25% by dry weight of the binder solution.
in which the acid precursor of the binder solution derivable from an inorganic salt comprises an ammonium salt.
in which the acid precursor of the binder solution derivable from an inorganic salt comprises an ammonium salt and in which the inorganic ammonium salt makes up between 5% and 25% by dry weight of the binder solution.

48. The method of claim 1 comprising at least one of the following features:
in which the acid precursor derivable from an inorganic salt of the binder solution comprises a species selected from the group consisting of sulphates, phosphates, nitrates and carbonates.
in which the reducing sugar of the binder solution has a dextrose equivalent value of at least 0.85.
in which the reducing sugar of the binder solution consists essentially of dextrose.
in which the binder solution comprises a silicon containing compound.

49. The method of claim 1 in which the binder solution comprises a material selected from the group consisting of a polycarboxylic acid, a salt of a polycarboxylic acid, an anhydride of a polycarboxylic acid.

50. The method of claim 1 comprising at least one of the following features:
in which the binder solution consists essentially of an aqueous solution of: a reducing sugar; at least one acid precursor derivable from an ammonium salt selected from the group consisting of ammonium sulphate salts, ammonium phosphate salts, and ammonium carbonate salts; and, optionally, excess ammonia.
in which the binder solution consists essentially of an aqueous solution of: a reducing sugar; at least one acid precursor derivable from an ammonium salt selected from the group consisting of ammonium sulphate salts and ammonium phosphate salts; and, optionally, excess ammonia, the binder solution having a pH which, in its conditions of use, prevents precipitation of sulphates or phosphates.
in which the binder solution consists essentially of an aqueous solution of: a reducing sugar; an acid precursor derivable from an ammonium salt; a carboxylic acid or a precursor thereof, and, optionally, excess ammonia.

51. The method of claim 1 comprising at least one of the following features:
in which the binder solution when sprayed on to the mineral fibres comprises at least 5% solids.
in which the binder solution when sprayed on to the mineral fibres comprises less than 50% solids.
in which the ratio by dry weight of reducing sugar to inorganic acid precursor expressed as (dry weight of reducing sugar/dry weight of inorganic acid precursor) is in the range 2.5 to 13.
in which the binder solution comprises between 0.1% and 1% of a silane or silicon-containing coupling agent calculated as dissolved binder solids.

52. The method of claim 1 comprising one of the following features:
in which the thermal insulation product has a thermal conductivity λ in the range 0.02 W/mK to 0.05 W/mK.
in which the thermal insulation product comprises glass wool fibres and has a density in the range of 8 to 50 kg/m³.
in which the thermal insulation product comprises stone wool fibres and has a density in the range of 25 to 180 kg/m³.

53. A mineral fibre thermal insulation product comprising mineral fibres and a binder, in which the binder is substantially formaldehyde free and comprises a reducing sugar; an acid precursor derivable from an inorganic salt and a source of nitrogen.

54. The mineral fibre thermal insulation product of claim 53 comprising at least one of the following features:
in which the binder has a pH of greater than 6 when dissolved in water.
in which the acid precursor of the binder derivable from an inorganic salt comprises an ammonium salt.
in which the acid precursor derivable from an inorganic salt of the binder comprises a species selected from the group consisting of sulphates, phosphates, nitrates and carbonates.

55. The mineral fibre thermal insulation product of claim 53 comprising at least one of the following features:
in which the reducing sugar of the binder has a dextrose equivalent value of at least 0.85.
in which the reducing sugar of the binder consists essentially of dextrose.

56. The mineral fibre thermal insulation product of claim 53 comprising at least one of the following features:
in which the binder comprises a silicon containing compound.
in which the binder comprises a material selected from the group consisting of a polycarboxylic acid, a salt of a polycarboxylic acid, an anhydride of a polycarboxylic acid.

57. The mineral fibre thermal insulation product of claim 53 comprising at least one of the following features:
in which the binder consists essentially of a reducing sugar; at least one acid precursor derivable from an ammonium salt selected from the group consisting of ammonium sulphate salts, ammonium phosphate salts, and ammonium carbonate salts; and, optionally, excess ammonia.
in which the binder consists essentially of a reducing sugar, an acid precursor derivable from an ammonium salt, a carboxylic acid or a precursor thereof and, optionally, excess ammonia.

58. The mineral fibre thermal insulation product of claim 53 comprising at least one of the following features:
in which the ratio by dry weight of reducing sugar to inorganic acid precursor expressed as (dry weight of reducing sugar/dry weight of inorganic acid precursor) is in the range 2.5 to 13.
in which the binder comprises between 0.1% and 1% of a silane or silicon-containing coupling agent calculated as binder solids.
in which the binder comprises glucosylamine.

59. A mineral fibre thermal insulation product obtainable by thermally curing a product in accordance with claim 53.

60. A mineral fibre thermal insulation product comprising mineral fibres and a binder, in which the binder is substantially formaldehyde free and comprises Maillard reaction products and in which the insulation product comprises more than 500 mg/kg of species selected from the group consisting of sulphates, phosphates, nitrates and carbonates.

61. The mineral fibre thermal insulation product of claim 60 comprising one of the following features:
in which the species selected from the group consisting of sulphates, phosphates, nitrates and carbonates is derived essentially from binder precursors.
in which the Maillard reaction products comprise melanoidins.

62. The mineral fibre thermal insulation product of claim 60 comprising one of the following features:
in which the thermal insulation product has a thermal conductivity λ in the range 0.02 W/mK to 0.05 W/mK.
in which the thermal insulation product comprises glass wool fibres and has a density in the range of 8 to 50 kg/m³.
in which the thermal insulation product comprises stone wool fibres and has a density in the range of 25 to 180 kg/m³.
in which the thermal insulation product comprises between 1% and 20% binder by dry weight.
